(19) United States
(12) Patent Application Publication
(10) Pub. No.: US 2010/0304500 A1
(43) Pub. Date: Dec. 2, 2010

(54) NANO-BIOSENSOR FOR BIOMOLECULAR RECOGNITION AND A METHOD OF SYNTHESIZING THE SAME

(76) Inventors: ELHAM MOHAGHEGHPOUR, TEHRAN (IR); FATOLLAH MOZTARZADEH, TEHRAN (IR); MOHAMMAD RABIEE, TEHRAN (IR)

Correspondence Address: CHOOBIN & CHOOBIN CONSULTANCY L.L.C;Barry Choobin, Green Community, Building 3, Ground Floor, Dubai Investment Park, P.O Box 212880, Dubai, AE

(21) Appl. No.: 12/826,726

(22) Filed: Jun. 30, 2010

(51) Int. Cl. G01N 33/566

(52) U.S. Cl. 436/501

(57) ABSTRACT

The various embodiments herein provide a nano-biosensor for detecting avidin bio-conjugated antibodies and a method of manufacturing the same. The nano-biosensor comprises a core made up of Zns: Mn nanoparticles. The core is surrounded by mercaptoelthanol molecules. Biotin is attached to the mercaptoethanol molecules surrounding the core. The ZnS:Mn nano particles with a size of 5-10 nm are prepared by quaternary W/O micro-emulsion method. According to one embodiment, a nano-biosensor comprises a nano particle of ZnS:Mn wherein the nano particle of ZnS:Mn includes ZnSO₄.7H₂O, Mn (NO₃)₂.4H₂O with different concentrations, Sodium sulphate, cyclohexane, Triton X-100, n-hexanol, sodium hydroxide, mercaptoethanol, thioglycolic acid and biotin.

### BACKGROUND

1. Technical Field

The embodiments herein generally relate to the field of bio molecular recognition and particularly a method of bio molecular recognition based on light scattering of colloidal semiconductor nano particle quantum dots such as nano particles functionalized with biotin. The embodiments herein more particularly relate to a nano-biosensor with phosphorescent nano particles for bio molecular recognition of avidin in biological media and a method of synthesizing the same.

2. Description of the Related Art

Biomolecular recognition has become an indispensable tool in clinical diagnostics as well as in pharmacology. Practically all relevant recognition schemes rely on some specific bio molecular detecting reactions. The integration of nanotechnology with biology and medicine has lead to major advances in molecular diagnostics, therapeutics, molecular biology and bioengineering.

Recent advances have led to the development of functional nanoparticles in fields like electronics, optical, magnetic, structural engineering, etc. These nanoparticles can covalently link to a biological molecule such as peptides, proteins or nucleic acids. Due to their size dependent properties to bio-macromolecules and dimensional similarities with these bio-macromolecules, these nano-bioconjugates have become well suited as contrasting agents for in vivo Magnetic Resonance Imaging (MRI), as long-circulating carriers for drug release/delivery and as structural scaffolds for tissue engineering. In addition, metal and semiconductor colloidal nanoparticles are under intensive study for potential applications in material synthesis, in multiplexed bioassays and in ultrasensitive optical detection and imaging.

Small organic dyes commonly used for diagnostic applications and in biological imaging had characteristics that limited their effectiveness. Problems with organic fluorescent markers include narrow excitation bands and broad emission spectra. This can make detection of multiple light emitting probes difficult because of spectral overlap, low resistance to chemical degradation and photo degradation.

Colloidal semiconductor nanoparticle Quantum Dots (QDs) are luminescent inorganic fluorophores which comprise of following processes: absorption, excitation, energy transfer and emission. Colloidal semiconductor nanoparticle Quantum Dots have the potential to overcome some of the functional limitations encountered by organic dyes in fluorescence labeling applications.

Luminescence emission from QD is detected at concentrations comparable to organic dyes by using conventional fluorescence methods, and individual QDs and QD-bio-conjugates are easily observable by microscopy.

ZnS is a typical II-VI semiconductor which has been commercially used as a phosphor as well as in thin film electroluminescent devices. There are many methodologies available for synthesizing ZnS nanocrystals (QDs) such as laser ablation, electrochemical fabrication, solvo-thermal and sol-gel methods. But these methods are time consuming and demand extreme pressure or temperature control. They are difficult to handle and require special or expensive equipments.

Hence there is a need for a method of synthesizing ZnS nano-crystal quantum dots which is simple, fast, efficient, easy to handle and does not demand extreme temperatures.

The above mentioned shortcomings, disadvantages and problems are addressed herein and will be understood by reading and studying the following specification.

### OBJECTIVES OF THE INVENTION

The primary object of the embodiments herein is to prepare biotinylated ZnS bio-sensors doped with manganese ions which can be used for detecting avidin bio-conjugated antibodies.

Another object of the embodiments herein is to provide a facile method of producing a biosensor which can be used as a common diagnostic reagent.

Yet another object of the embodiments herein is to prepare biotinylated ZnS bio-sensors doped with manganese ions which can be used for a wide range of diagnostic applications.

Yet another object of the embodiments herein is to provide a novel method of preparing ZnS: Mn nanoparticle bio-sensor functionalized with biotin for recognition of bio-molecules based on light scattering phenomenon.

Yet another object of the embodiments herein is to synthesis a Mn doped ZnS nanoparticles using the quaternary W/O micro-emulsion systems.

Yet another object of the embodiments herein is to synthesis ZnS: Mn nanoparticles having a distinguishable morphology of quaternary w/o micro-emulsion systems.

Yet another object of the embodiments herein is to synthesis a ZnS: Mn nanoparticles in which ions are better connected to each other.

Yet another object of the embodiments herein is to use reverse micelles technique wherein the water drops surround the nanocrystal and get imprisoned in the oil bulk so that these micelles can perform as nano-scaled reactors.

These and other objects and advantages of the embodiments herein will become readily apparent from the following detailed description taken in conjunction with the accompanying drawings.

### SUMMARY

The various embodiments herein provide a nano-biosensor for detecting avidin bio-conjugated antibodies. According to one embodiment herein the nano-biosensor comprises of a core made up of ZnS and Mn nanoparticles. The core is surrounded by a linking agent. A biological part is attached to the linking agent surrounding the core. The ZnS and Mn nanoparticles are prepared by quaternary W/O micro-emulsion method. The size of the ZnS and Mn nanoparticles is in the range of 5-10 nm. The linking agent herein used is mercaptoethanol and the biological part is biotin.

According to another embodiment herein, a method of synthesizing a nano-biosensor for recognizing avidin bio-conjugated antibodies. ZnS and Mn nanoparticles are prepared by quaternary micro-emulsion method. Mercaptoethanol as linking agent is added. Biotin as biological part is added.

According to another embodiment herein, the steps for preparing ZnS and Mn nanoparticles involve preparing micro-emulsion solution of cyclohexane, Triton X-100 and n-hexonal, dividing the solution in two equal halves in two separate beakers, preparing aqueous stock solutions of (0.1 M) ZnSO₄.7H₂O, (5.5%) Mn (NO₃)₂.4H₂O and (0.1 M) Na₂S, adding 10 cc of the aqueous stock solution of ZnSO₄.7H₂O and Mn (NO₃)₂.4H₂O to the first beaker, adding 10 cc of the aqueous stock solution of Na₂S to another beaker, vigorously agitating the mixtures by a magnetic stirrer for 15-120 min, mixing the two separate mixtures and further agitating for 15 min, keeping the resultant mixture for 2 days at room temperature for the rest of the aging time and adding mercaptoethanol and biotin to the final solution.

According to another embodiment herein, cyclohexane is used as oil phase, Triton X-100 is used as surfactant and n-hexnal is used as co-surfactant.

According to one embodiment herein, a nano-biosensor for bio molecular recognition comprising a nano particle of ZnS: Mn wherein the nano particle of ZnS: Mn comprises Zinc Sulphate hepta hydrate (ZnSO₄.7H₂O), Manganese nitrate tetrahydrate (Mn (NO₃)₂.4H₂O), Sodium sulphate, an oil, surfactant, co-surfactant, sodium hydroxide, linking agent and biotin.

The oil is cyclohexane. The surfactant is triton X-100. The co-surfactant is n-hexonal. The ratio of surfactant and co-surfactant is 1:1. The linking agents are mercaptoethanol and thioglycolic acid. The linking agent is mercaptoethanol. The Mn(NO₃)₂.4H₂O has different concentrations and the different concentrations are 0.01%, 0.7%, 2%, 3%, 5.5%, 9% and 13%. The size of nano particles of ZnS:Mn is 5-10 nm.

According to another embodiment, a method of synthesizing a nano-biosensor for biomolecular recognition comprises preparing a micro-emulsion solution by mixing cyclohexane, Triton X-100 and n-hexonal. The micro-emulsion solution is transferred into a first beaker and second beaker so that the amount of the micro-emulsion solution in the first beaker and the amount of the micro-emulsion solution in the second beaker are equal. An aqueous stock solution of ZnSO₄.7H₂O with a molarity of 0.1 is prepared. A pluralities of aqueous stock solutions of Mn(NO₃)₂.4H₂O with different concentrations of 0.01%, 0.7%, 2%, 3%, 5.5%, 9% and 13% are prepared. An aqueous stock solution of Na₂S with a molarity of 0.1 is prepared.

The aqueous stock solution of ZnSO₄.7H₂O and the pluralities of aqueous stock solutions of Mn (NO₃)₂.4H₂O with different concentrations are added to the contents of first beaker. The content of the first beaker is agitated vigorously by a magnetic stirrer for 15-120 min after adding the aqueous stock solution of ZnSO₄.7H₂O and the pluralities of aqueous stock solutions of Mn(NO₃)₂.4H₂O with different concentrations.

The aqueous stock solution of Na₂S is added to the contents of the second beaker. The content of the second beaker is agitated by a magnetic stirrer for 15-120 min after adding the aqueous stock solution of Na2S. The content of the first beaker and the content of the second beaker are mixed after the agitating process to obtain a resultant mixture. The resultant mixture is agitated for 15 min and kept at room temperature for 2 days for aging.

A linking agent is added to the resultant mixture after aging. A biological agent is added to the resultant mixture to obtain a nano particle of ZnS: Mn. The nano particle of ZnS: Mn is prepared by quaternary w/o micro-emulsion method. The cyclohexane is used as oil phase. The Triton X-100 is used as surfactant. N-hexanol is used as co-surfactant. The linking agents are mercaptoethanol and thioglycolic acid. The linking agent is mercaptoethanol. The biological agent is biotin.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows schematic diagram of the ZnS:Mn nano-biosensor conjugated with biological part with the help of a linking agent.

FIG. 2 shows a flow chart illustrating the steps of synthesizing ZnS:Mn phosphorescent nano-particles by quaternary micro-emulsion method, according to one embodiment here in.

FIG. 3 illustrates a schematic presentation of ZnS:Mn nano-crystal or micelle during the synthesizing of ZnS:Mn phosphorescent nano-particles by quaternary micro-emulsion method, according to one embodiment here in.

FIG. 4 illustrates the synthesis of ZnS:Mn nanoparticles showing the conjugation of biotin to the ZnS:Mn nanocrystal, during the synthesizing of ZnS:Mn phosphorescent nano-particles, according to one embodiment here in.

FIG. 5 shows the scanning electron micrograph (SEM) of ZnS:Mn nano particle according to one embodiment here in, with a indicating the scanning electron micrograph is at low magnification and b indicating the scanning electron micrograph is at high magnification.

FIG. 6 shows transmission electron microscopy (TEM) images of ZnS:Mn nanoparticle prepared by using micro-emulsion method according to one embodiment here in, with a and b indicating the various TEM images of ZnS:Mn nanoparticles.

FIG. 7 shows an energy dispersive X-ray spectroscopy (EDS) diagram of one point of nanoparticle powder of ZnS:Mn nano-biosensor according to an embodiment.

FIG. 8 shows EDS diagram of nanoparticle powder of ZnS as in the prior art.

FIG. 9 shows an emission spectra intensities of ZnS:Mn nanocrystal, with a black curve indicating the intensity of ZnS:Mn nanocrystal with a linking agent, a grey curve indicating the intensity of ZnS:Mn nanocrystal with mercaptoethanol and light grey curve indicating the intensity of ZnS:Mn nanocrystal with thioglycolic acid.

FIG. 10 shows a comparison of zeta potential of ZnS: Mn nano-particles linked without or with linking agents with a indicating the zeta potential for ZnS: Mn nano-particles without linking agent, b indicating the zeta potential for ZnS: Mn nano-particles linked with mercaptoethanol and c indicating the zeta potential for ZnS: Mn nano-particles linked with thioglycolic acid.

FIG. 11 shows the photoluminescence spectra of ZnS: Mn nanoparticles doped with different concentrations of Mn²⁺ ions at room temperature at 321 nm.

FIG. 12 shows the energy band diagram indicating the excitation of the ZnS: Mn nanoparticle.

FIG. 13 shows the emission spectra of ZnS: Mn nanoparticles with different concentrations of biotin in biological media. Here Ml is microlitre.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a nano-biosensor for detecting avidin bio-conjugated antibodies. A biosensor is an analytical device that uses biological molecules to detect other biological molecules or chemical substances. To be a suitable bio-labeling agent (biosensor), the nanoparticles should have high luminescent efficiency and proper surface groups for coupling with biomolecules.

Avidin is a tetrameric protein produced in the oviducts of birds, reptiles and amphibians deposited in the whites of their eggs. Avidin has a high degree of affinity and specificity to biotin. This has resulted in a great number of applications of avidin-biotin interaction as a common diagnosis reagent in fields of biochemistry, immunoassays, receptor and histochemical studies, bacteriophage inhibitions, etc.

According to one embodiment herein, the nano-biosensor comprises of a core made up of ZnS and Mn nanoparticles. The core is surrounded by a linking agent. A biological part is attached to the linking agent surrounding the core. The ZnS and Mn nanoparticles are prepared by quaternary W/O micro-emulsion method. The size of the ZnS and Mn nanoparticles is in the range of 5-10 nm. The linking agent herein used is mercaptoethanol and the biological part used is biotin.

ZnS is a typical II-VI semiconductor, especially if doped with divalent manganese ions. It has been commercially used as a phosphor as well as in thin film electroluminescent devices. ZnS nanocrystal QDs has size-dependent tunable photoluminescence (PL) with broad excitation spectra and narrow emission bandwidths (full width at half maximum of ˜30-45 nm) that span the visible spectrum. This allows a simultaneous excitation of several particle sizes at a single wavelength. In addition, ZnS nano-crystal QDs have a high photochemical stability, an excellent resistance to chemical degradation and photo degradation and a good fluorescence quantum yield. These, when utilized with antibodies are successful in the detection of the protein toxins like staphylococcal enterotoxin B, cholera toxin, etc.

The biological part cannot be connected to the nano-crystal without an inter-mediator. Thus, a linking agent gives ability to nano-crystal for conjugating with the biological part. Thus, ZnS: Mn nanoparticle is surrounded with the linking agent and then this particle is connected to the biological part. According to various embodiments herein, mercaptoethanol is used as linking agent. The schematic formulation of alcoholic nanoparticle coated with mercaptoethanol is as follows:

HOCH₂CH₂S(ZnS)SCH₂CH₂OH.

When the nano-sensor is coupled with a biomolecule such as avidin, the luminescence spectra characterization is influenced. So, when the avidin concentration increases, the emission decreases. Thus, this material can be used for recognition of avidin conjugated biomolecules because of its selective influence on elements in biologic medium and can detect avidin concentration by measuring the emission-decreasing rate.

Biotin is a water-soluble B-complex vitamin (vitamin B₇) that is composed of an ureido (tetrahydroimidizalone) ring fused with a tetrahydrothiophene ring. Biotinylation is the process of covalently attaching a biotin molecule to a molecule or surface. This biotinylated tag can be used in detection of the protein via avidin tagged detectors. The biotin-avidin system plays a major role in the field of bio-molecular recognition as it exhibits highly specific and strong binding affinity.

According to one embodiment herein, a method of synthesizing a nano-biosensor for recognizing avidin bio-conjugated antibodies. ZnS and Mn nanoparticles are prepared by a quaternary micro-emulsion method. A micro-emulsion can be characterized as oil-in water (O/W), water-in-oil (W/O) or bi-continuous system. The oil-in-water is a micro-emulsion containing an excess oil phase with surfactant molecules existing in the aqueous phase in form of normal micelles. On the other hand, the water-in-oil (W/O) micro-emulsion is the coexistence of an excess water phase and the surfactant molecules which aggregate in the oil phase in the form of reverse micelle. The Water-in-oil (W/O) micro-emulsions or reverse micelles technique is one of the most widely recognized methods due to its several advantages, for instance, soft chemistry, demanding no extreme pressure or temperature control, easy to handle and requiring no special or expensive equipment. In general, micro-emulsion or ME is an isotropic, thermodynamically stable dispersion of oil, water, surfactant and often co-surfactant, which is normally alcohol.

According to one embodiment herein, ZnS and Mn nanoparticles are prepared by the quaternary micro-emulsion method, wherein cyclohexane is used as oil phase, Triton X-100 as surfactant, n-hexanol as a co-surfactant. A surfactant and a co-surfactant produce a stable emulsion which facilitates a connection to the surface of the nano particles and prevents from contacting together. The surfactants and the co-surfactants lower the surface tension of a liquid, allowing easier spreading and lowering of the interfacial tension between two liquids. To prepare the ZnS and Mn nanoparticles; firstly, a solution of cyclohexane (67.32 gr), Triton X-100 (1.29 gr) and n-hexanol (2 gr) is prepared. The solution is divided in two equal halves in two separate beakers. The aqueous stock solutions of (0.1 M) ZnSO₄.7H₂O, (5.5%) Mn(NO₃)₂.4H₂O and (0.1 M) Na₂S are prepared. 10 cc of the aqueous stock solution of ZnSO₄.7H₂O and Mn(NO₃)₂.4H₂O are added to the first beaker. 10 cc of the aqueous stock solution of Na₂S is added to another beaker. The mixtures are vigorously agitated by a magnetic stirrer for 15-120 min. The two separate mixtures are mixed and further agitated for 15 min. The resultant mixture is kept for 2 days at room temperature for the rest of the aging time. Mercaptoethanol and biotin are added to the final solution.

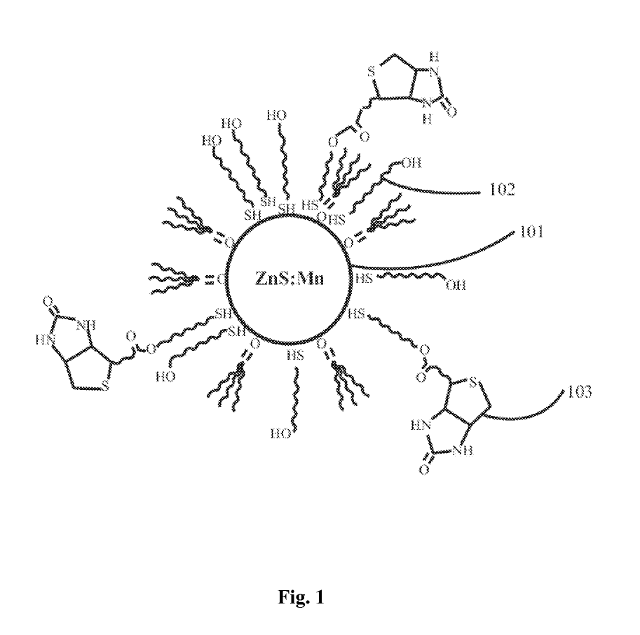

FIG. 1 shows a schematic diagram of the ZnS: Mn nano-biosensor conjugated with biological part with the help of a linking agent. With respect to FIG. 1, ZnS: Mn nanocrystal 101 is surrounded by the molecules of the linking agent 102. The linking agent herein is Mercaptoethanol. The linking agent gives the ability to a nanocrystal to conjugate with biological part. The biological part 103 gets attached to the linking agent. The biological part herein is biotin. This biotin gets attached to avidin, thereby reducing the concentration of avidin. The microlitre change in concentration of avidin results in change in the emission spectra. Thus the nano-biosensor can be used to detect various avidin conjugated antibodies.

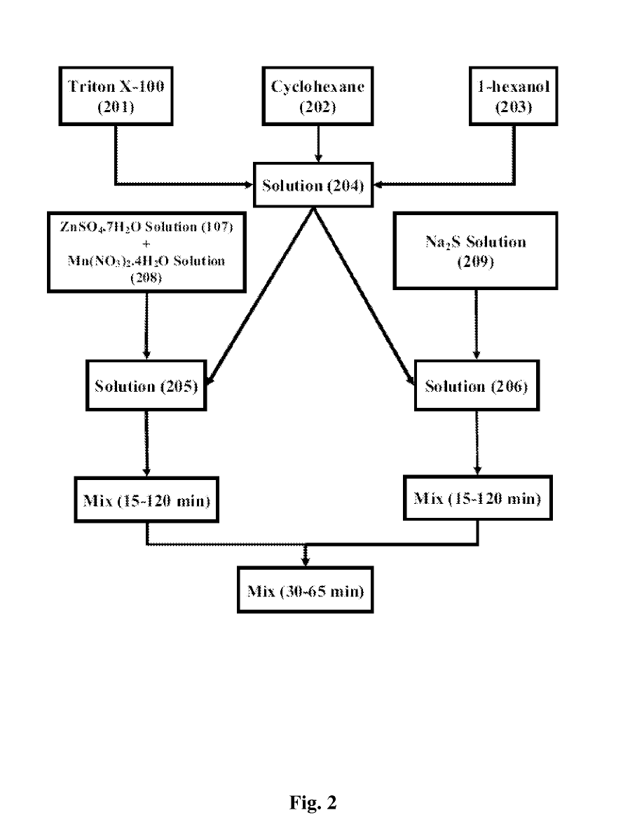

FIG. 2 shows a flow chart illustrating the general stages of synthesis of ZnS: Mn phosphorescent nanoparticles by quaternary micro-emulsion method. With respect to FIG. 2, a solution 204 is prepared by mixing 1.29 gr of Triton X-100 201, 67.32 gr of cyclohexane 202 and 2 gr of n-hexanol 203. The solution 204 is separated in two equal halves 205 and 206 in two accurate beakers. An aqueous stock solution of 0.1 M ZnSO₄.7H₂O 207 is prepared. Stock solution of 5.5% Mn (NO₃).4H₂O 208 is prepared. 10 cc of stock solution 207 and 10 cc stock solution 208 are added into the first beaker containing solution 205. Aqueous stock solution in the concentration of 0.1M Na₂S 209 is prepared.10 cc of stock solution 209 is added into micro-emulsion solution 206 contained in the second beaker. Finally, the emulsions are vigorously agitated by a magnetic stirrer for about 15-120 min. The two separate micro-emulsion solutions 205 and 206 are mixed together and agitated about a further of 15 min. The resulting mixture is then allowed to stand for 2 days at room temperature for the rest of the aging time. After ZnS: Mn nanoparticles are synthesized; mercaptoethanol is added and then is added biotin.

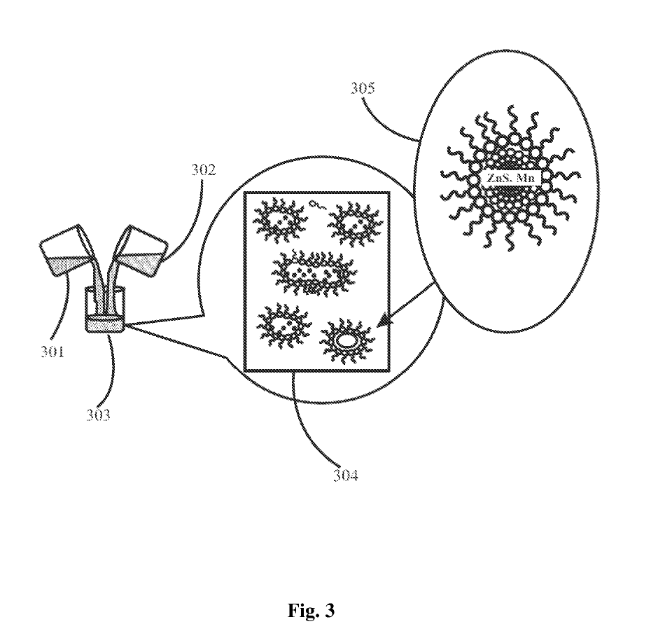

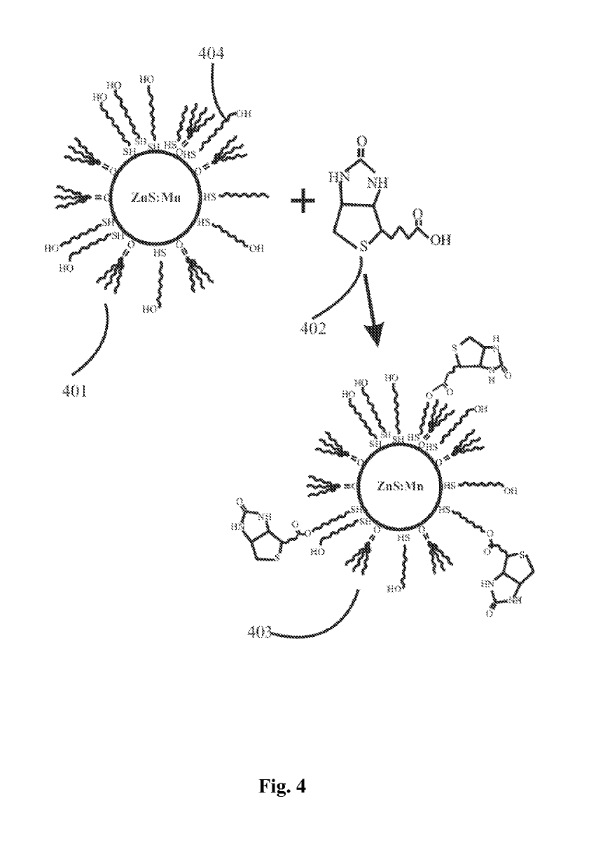

FIG. 3 illustrates a schematic presentation of ZnS: Mn nano-crystal or micelle. With respect to FIG. 3, solution 301 prepared by adding 10 cc ZnSO₄.7H₂0 and 10 cc Mn (NO₃)2.4H₂O to a micro-emulsion solution made of Triton X-100, cyclohexane and n-hexanol, is mixed with the solution 302 prepared by adding 10 cc Na₂S to micro-emulsion solution made of Triton X-100, cyclohexane and n-hexanol. The resultant solution 303 formed is agitated. The following reaction 304 takes place:

ZnSO₄+Na₂S→ZnS+Na₂SO₄

During the agitation process, the water molecules that surround the nano-crystal get imprisoned in the oil bulk of micro-emulsion solution. The micelle 305 formed acts as nano-scaled reactor.

The nano particles made by other methods tend to aggregate within a first few minutes and their size become larger. This is due to their large surface to volume ratio. To avoid these problems, reverse micelles technique is applied, wherein the water drops that surround the nano-crystal get enclosed in the oil bulk phase. Thus, allowing these micelles to perform as nano-scaled reactors.

FIG. 4 illustrates the synthesis of ZnS:Mn nanoparticles showing the conjugation of biotin to the ZnS:Mn nanocrystal. With respect to FIG. 4, the ZnS: Mn nanocrystal micelle 401 has mercaptoethanol molecules 404 acting as a linking agent. The ZnS: Mn nanocrystal micelle is linked with biotin 402. After linking with biotin 402, a Biotin-Mer-Qd complex 403 is formed, wherein the biotin molecules are attached to the ZnS: Mn micelle with the help of linking agent, herein mercaptoethanol.

### Experimental Data

First, the solution of cyclohexane, Triton X-100 and n-hexanol was prepared and mixed in two accurate beakers. Then, the aqueous stock solutions (0.1 M) of ZnSO₄.7H₂O and Mn(NO₃)₂.4H₂O with different concentrations (0.01%, 0.7%, 2%, 3%, 5.5%, 9% and 13%) were added into the first beaker and Na₂S (0.1 M) was added into micro-emulsion solution in another beaker. Finally, the emulsions were vigorously agitated by a magnetic stirrer. After mechanical agitation for about 15-120 min, two separate micro-emulsion solutions were mixed together and agitated about a further 15 min. The resulting mixture was then allowed to stand for 2 days at room temperature for the rest of the aging time.

Herein, the reverse micelles technique was used, wherein the water drops that surrounded the nanocrystal were imprisoned in the oil bulk and these micelles could perform as nano-scaled reactors. The effect of Mn concentration on the emission spectra was studied. Also, the effect of two different types of linking agent on the emission spectral intensity of ZnS:Mn was evaluated.

Table 1 lists all the reagents required for ZnS:Mn nano-powder synthesis. These reagents are used as received without any further purification from Merck Company.

TABLE 1
THE REAGENTS USED FOR POWDER SYNTHESIS

| Product name | Chemicals | Molar mass(gr) | Density (g/cm³) |
|---|---|---|---|
| Zinc sulfate | ZnSO₄•7H₂O | 287.54 | 1.97 |
| heptahydrate | | | |
| Sodium sulfate | Na₂S | 78.04 | 1.43 |
| Manganese | Mn(NO₃)₂•4H₂O | 251.01 | 2.13 |
| nitrate | | | |
| tetrahydrate | | | |
| Triton X-100 | C₈H₁₇C₆H₄ | n = 10 | 1.07 |
| | (OCH₂CH₂)ₙOH | 646 | |
| cyclohexane | C₆H₁₂ | 84.169 | 0.78 |
| n-hexanol | CH₃(CH₂)₅OH | 102.18 | 0.82 |
| Sodium | NaOH | 40 | 2.13 |
| hydroxide | | | |
| mercaptoethanol | C₂H₅OH | 46.07 | 0.790-0.793 |
| mercaptoethanol | C₂H₆OS | 78.13 | 1.12 |
| thioglycolic | C₂H₄O₂S | 92.12 | 1.32 |
| Biotin | C₁₀H₁₆N₂O₃S | 244.31 | |

For qualitative and quantitative analyses of the embodiment herein Scanning Electron Microscopy (SEM), Transmission Electron Microscopy (TEM), Energy Dispersive X-ray Spectroscopy (EDS), Inductive coupled plasma (ICP), zeta meter for measurement ZP and spectrograph techniques were used.

As observed in the experiments, nano particles were made within the first few minutes, but after few minutes, those particles were aggregated and their size got larger because of their large surface to volume ratio. To avoid those problems, reverse micelles technique was used. In this method, water drops that surrounded the nano-crystal were enclosed in the oil bulk phase and these micelles could perform as nano-scaled reactors.

The biological part cannot be connected to the nano-crystal without inter-mediator. A linking agent was used to connect the biological molecule to the nano-crystal. Thus, linking agent gave ability to nano-crystal for conjugating with the biological part.

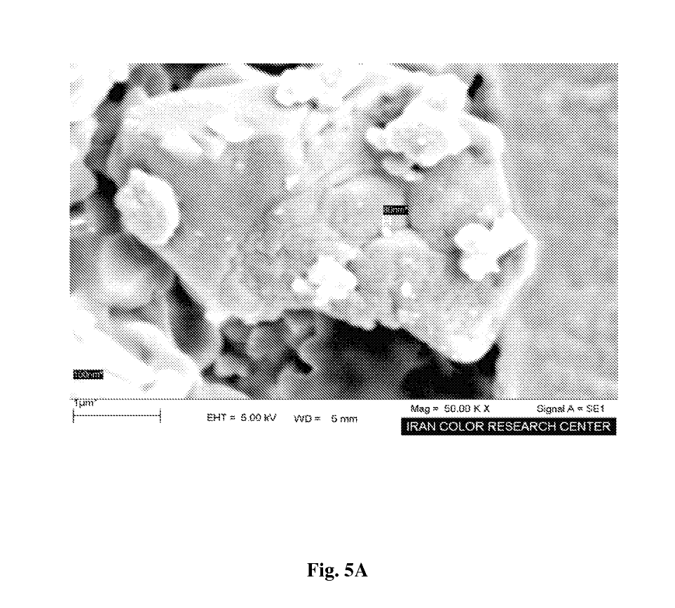

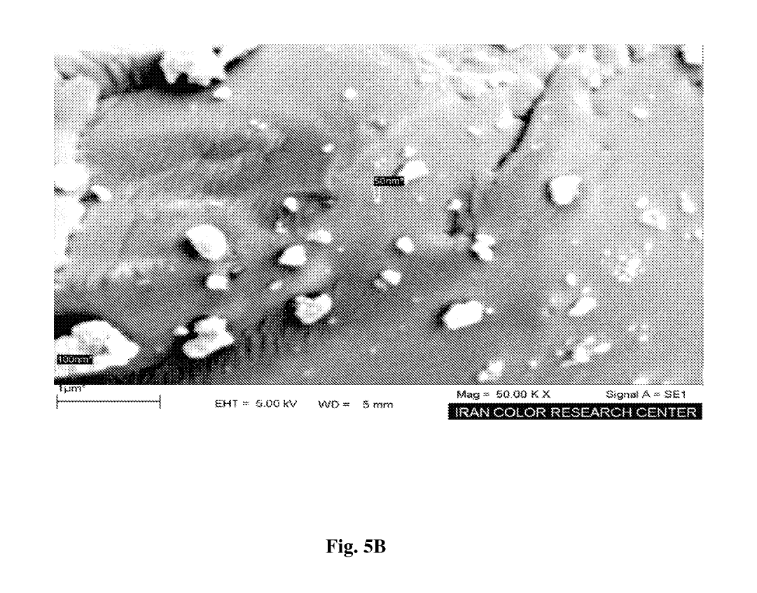

FIG. 5 shows the scanning electron micrograph (SEM) of ZnS:Mn nanoparticle, wherein a is at low magnification and b is at high magnification. Scanning Electron Microscopy (SEM) allows imaging of individual crystallites and the development of a statistical description of the size and shape of the particles in a sample. With respect to FIG. 5, the particle size of ZnS dry powder was less than 100 nm in diameter. The ZnS aggregated particles were composed of much smaller crystallites.

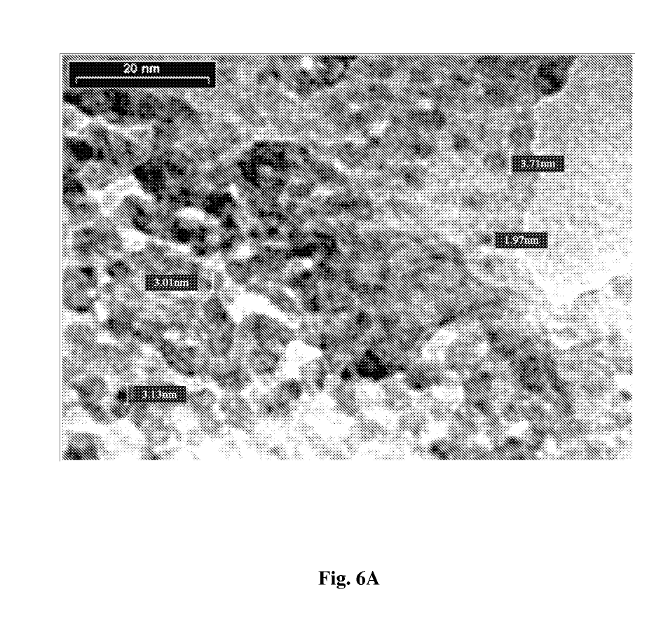

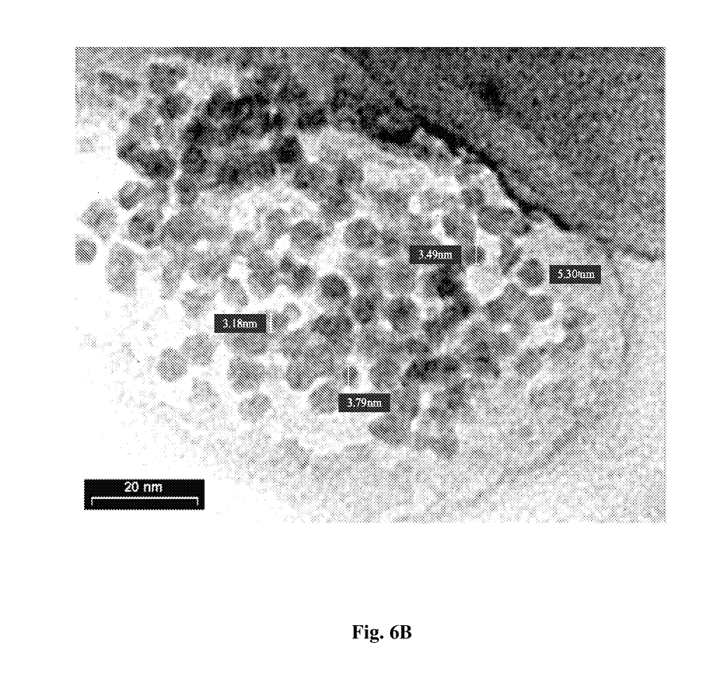

FIG. 6 shows the transmission electron microscopy (TEM) images of ZnS:Mn nanoparticle prepared by using micro-emulsion method, wherein a and b shows various TEM images of ZnS:Mn nanoparticles. TEM at high magnification imaging allows the determination of individual crystallite morphology. With respect to FIG. 6, a and b, it was found that the diameter of zinc sulfide particles is less than 5 nm. This is consistent with the particle size determined by using XRD analysis. The TEM observations confirmed that the nanoparticles have been formed by crystallite agglomeration.

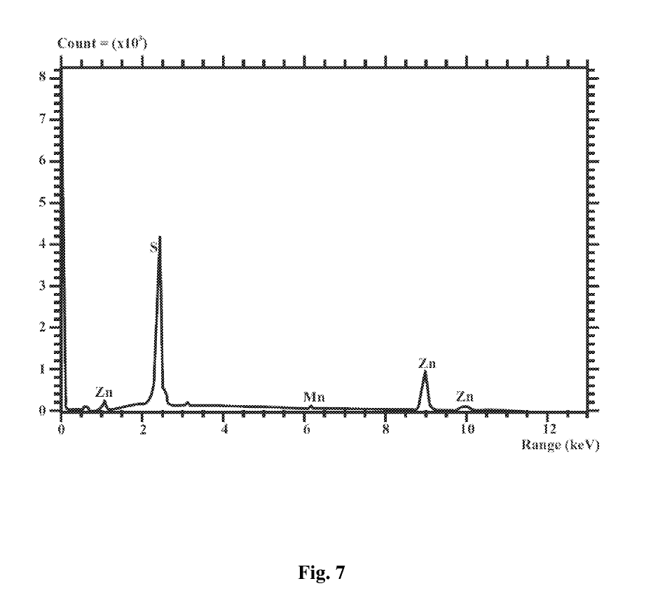

FIG. 7 shows an energy dispersive X-ray spectroscopy (EDS) diagram of one point of nanoparticle powder of ZnS:Mn nano-biosensor. With respect to FIG. 7, peaks at 1.0 and 2.3 keV exhibit the combination of Zn and S. Meanwhile, the smaller peaks at 8.6 and 9.6 keV correspond to the transition of Zn Ka and Kb respectively. The EDS analysis demonstrated that these products are ZnS:Mn nano-crystals.

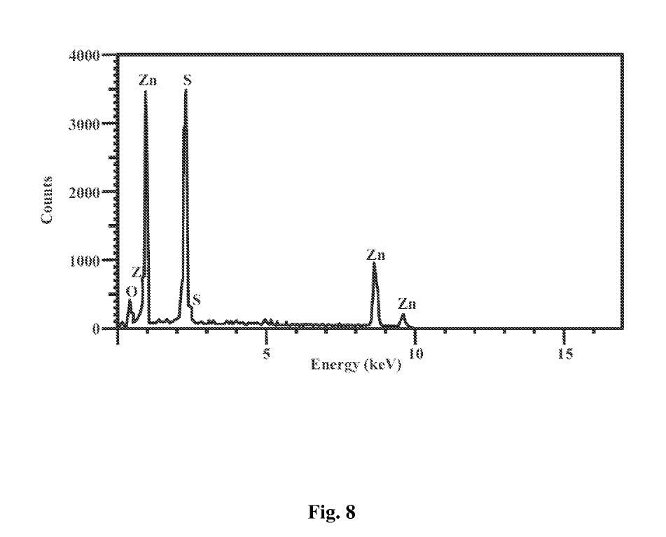

FIG. 8 shows EDS diagram of nanoparticle powder of ZnS as in the prior art. With respect to FIG. 8 and also FIG. 7, a comparison of the results with the prior art showed that peak boundaries were the same as that reported in the prior art. However, the peak height was less than that in prior art. In addition, another peak was also seen that showed a small amount of Mn in ZnS nano-crystal. Thus, this peak can be related to the replacement of some Zn⁺² ions by Mn⁺² in ZnS:Mn.

It is to be noted that the actual concentration of Mn²⁺ ions is different from the initial concentration. So, to determine the actual doping concentration of Mn²⁺ ion ICP analysis was performed. Table 2 shows the results of ICP tests showing the concentration of various elements contained in the synthesized nanocrystal.

TABLE 2
ICP TEST RESULTS OF ELEMENTS CONTAINED
IN SYNTHESIZED NANOCRYSTAL

| Material | Percent (%) |
|---|---|
| Na | 8.42 |
| S | 16.15 |
| Zn | 20.80 |
| Mn | 0.235 |

Mn²⁺ ion in a ZnS nanoparticle has two choices of its nearest cat ions. They are as follows: (i) Zn²⁺ and (ii) Mn²⁺. Since the actual Mn²⁺ concentration is low in the sample, it was found that these Mn²⁺ ions are surrounded by Zn²⁺ ions, therefore no Mn²⁺ pairs are formed. It is notable that the obtained results that were reported by ICP examination confirm the EDS diagram for low concentration of Mn²⁺ ion in the sample.

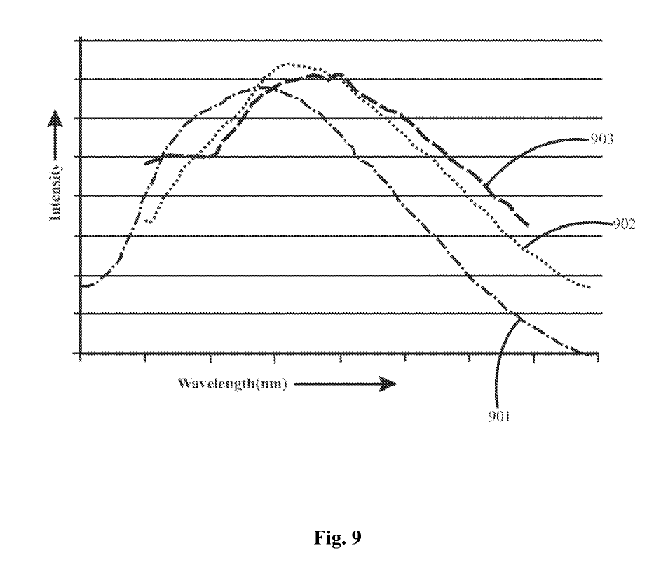

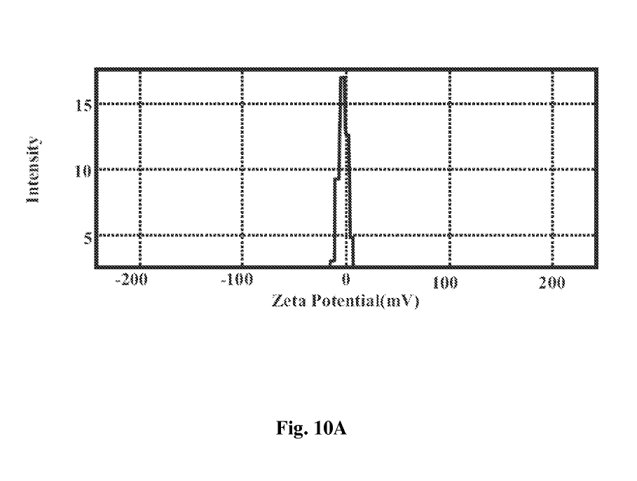

FIG. 9 shows an emission spectra intensities of ZnS:Mn nanocrystal, wherein black curve shows intensity of ZnS:Mn nanocrystal with a linking agent, grey curve shows intensity of ZnS:Mn nanocrystal with mercaptoethanol and light grey curve shows intensity of ZnS:Mn nanocrystal with thioglycolic acid. The schematic formulation of alcoholic nanoparticle coated with mercaptoethanol is as follows:

HOCH₂CH₂S(ZnS)SCH₂CH₂OH

### Mercaptoethanol

The schematic formulation of alcoholic nanoparticle coated with thighlycolic acid is as follows:

HOOCCH₂SH(ZnS)SHCH₂COOH

### Thioglycolic Acid

With respect to FIG. 9, the photoluminescence emission peak of nanoparticles with mercaptoethanol is approximately larger than thioglycolic acid. This shows that ZnS:Mn coated by phosphorous mercaptoethanol and biotin conjugated as biologic part was found to have much more affinitive to avidin. This is due to presence of the active hydroxyl groups.

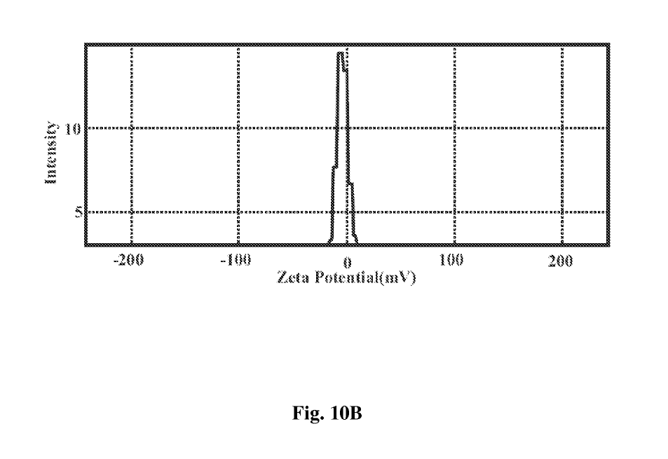

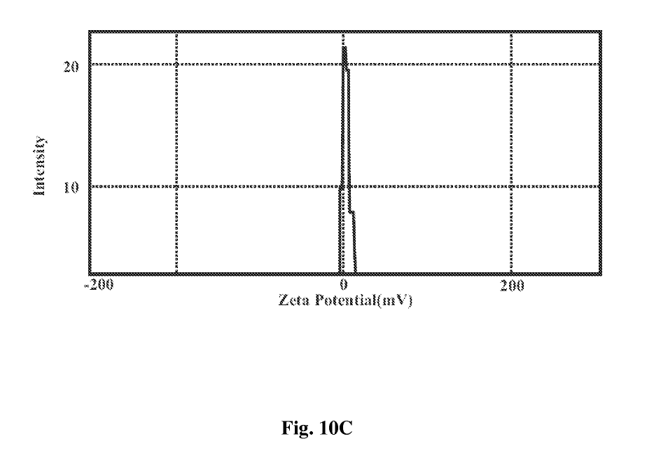

FIG. 10 shows comparison of zeta potential of ZnS:Mn nano-particles linked without or with linking agents wherein a shows zeta potential for ZnS:Mn nano-particles without linking agent, b shows zeta potential for ZnS:Mn nano-particles linked with mercaptoethanol and c shows zeta potential for ZnS:Mn nano-particles linked with thioglycolic acid. With respect to FIG. 10, the Zeta potential for ZnS:Mn nano-particles without linking agent came out to be −3.1 mV. The zeta potential for ZnS: Mn nano-particles linked with mercaptoethanol came out to be −4.8 mV. The zeta potential for ZnS:Mn nano-particles linked with thioglycolic acid came out to be +1.9 mV. Since a magnitude of the zeta potential gives an indication of the potential stability of the colloidal system, it can be seen that mercaptoethanol gave more stability to nanoparticles. Moreover, if all the particles in suspension have a large negative or positive zeta potential, then they will tend to repel each other and there is no tendency for the particles to come together and if the particles have low zeta potential values, then there is no force to prevent the particles coming together and flocculating.

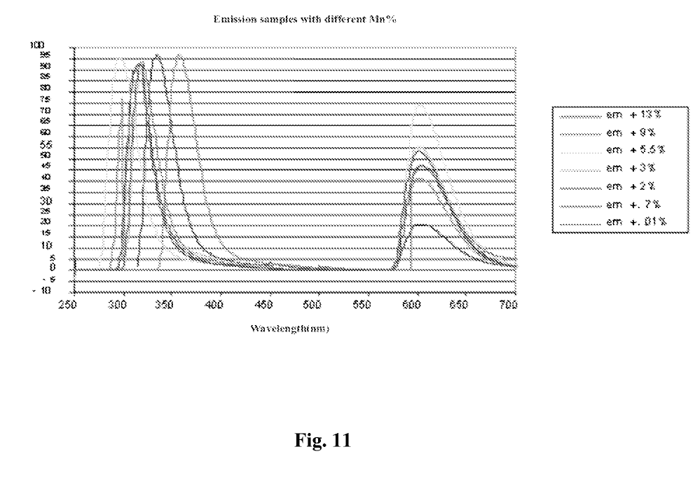

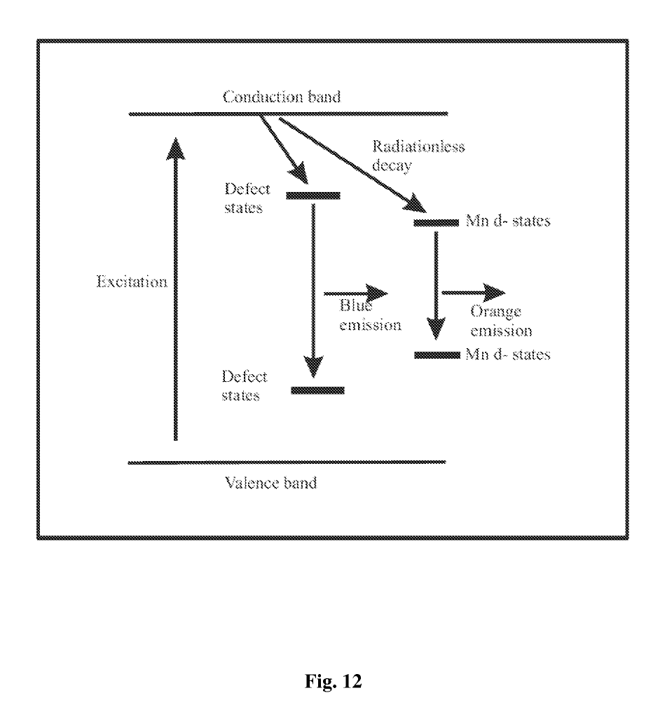

FIG. 11 shows the photoluminescence spectra of ZnS:Mn nanoparticles doped with different concentrations of Mn²⁺ ions at room temperature at 321 nm. With respect to FIG. 11, it can be seen that two different emission bands are dominating the PL spectra. The first emission band ranging from 290-360 nm is broad and can be assigned to the radiative recombination involving defect states in the ZnS nanocrystals as also will be suggested in the schematic diagram in FIG. 12 later. Also this emission band should indeed originate from the host ZnS but not from Mn²⁺ ions. The second emission band was centered at about 602 nm which was due to ⁴T₁→⁶A₁ transition within the 3d shell of Mn²⁺. When Mn²⁺ ions were incorporated into the ZnS lattice and substituted for host cation sites, the mixing between the s-p electrons of the host ZnS and the d electrons of Mn²⁺ occurred and made the forbidden transition of ⁴T₁→⁶A₁ partially allowed, resulting in the characteristic emission of Mn²⁺. It is known that Mn²⁺ incorporated into the ZnS lattice leads to the Mn²⁺ based orange emission while ZnS with surface-bound Mn²⁺ yields the ultraviolet emission. Thus, it can be concluded that the Mn²⁺ ions in our samples are indeed incorporated into the host ZnS nanocrystals. It is notable that the concentration quenching has been mainly attributed to the migration of the excitation energy between Mn²⁺ ion pairs in the case of Mn²⁺ doping. Thus, the existence of Mn²⁺ pairs is important for the occurrence of the concentration quenching effect. During the concentration quenching process, the excitation energy is transferred from one Mn²⁺ ion to its nearest Mn²⁺ ion by non-radiative transitions and via a number of transfer steps finally to a quenching site, for example, defect state. Many researchers have found quenching concentration for the photoluminescence intensity of Mn²⁺ doped ZnS nanocrystals. They are 2%, 0.12%, 1%, and 1.5%. According to one embodiment herein concentration quenching was also observed in the samples as shown in FIG. 11. It was found that the Mn²⁺⁴T₁→⁶A₁ emission intensity of Mn: ZnS d-dots significantly increased with the increase of Mn²⁺ concentration and showed a maximum when Mn²⁺ doping content was 5.5%. If Mn²⁺⁴T₁→⁶A₁ concentration continued to increase, namely more than 5.5%, the Mn²⁺⁴T₁→⁶A₁ emission intensity would decrease.

FIG. 12 shows the energy band diagram wherein shows the excitation of the ZnS:Mn nanoparticle. With respect to FIG. 12, an electron from the ZnS valence band is excited across the band gap and the photo excited electron subsequently decays by a normal recombination process to some surface or defect states. At higher Mn concentration, the isolated Mn ion may stay at the surface or interstitial positions of the crystallites with octahedral symmetry and these do not favor radiative transitions. As the highest Mn concentration in the power is more than 5.5%, Mn-Mn dipolar interactions may be predominant in the micro-emulsion solution. The emission intensity at 602 nm changes relative to the Mn⁺² emission intensity for different concentrations of Mn. This happens because at higher concentrations of Mn not all the Mn⁺² ion enters in the lattice in a substitution manner, but isolated Mn⁺² ions may stay at the surface or interstitial positions. Hence, although the intensity of the peak due to the sulfur vacancy decreases, this is not accompanied by an increase of Mn⁺² emissions.

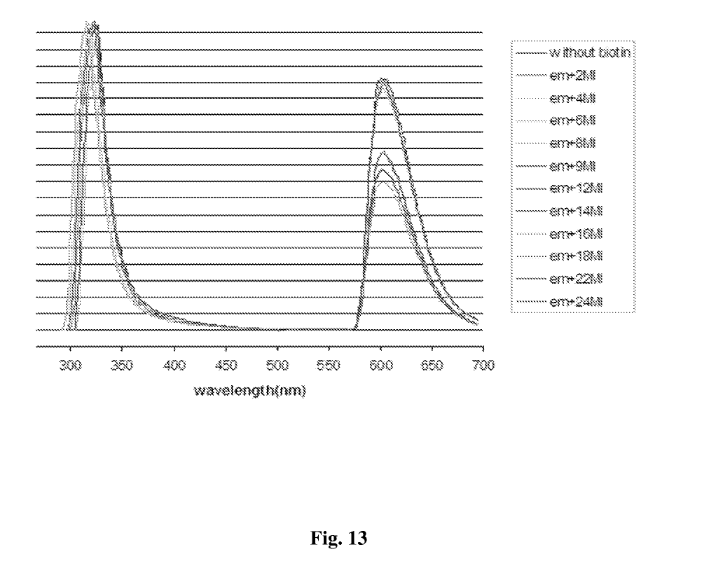

FIG. 13 shows the emission spectra of ZnS:Mn nanoparticles with different concentrations of biotin in biological media. Herein, Ml is microlitre. There were some significant changes in the morphology of the synthesized ZnS:Mn nanoparticle when it was coupled with Biotin. So titration of this particle with a biologic function such as Avidin could influence the luminescence spectra characterization of this particle and when avidin concentration increased, emission decreased intensively. With respect to FIG. 13, the highest photoluminescent intensity was measured in solution without biotin and the signal decreased proportionally with increasing of biotin, varying from 0 to 24 μL. The photoluminescence of the ZnS:Mn solution alone was not found to change significantly over the time of this experiment. But it was observed that adding a small amount of biotin in avidin biologic media could reduce emission significantly. As shown in FIG. 13, with the increase of biotin to nanocrystal micro-emulsion media to concentration more than 18 μL, no changes had been seen in the emission intensity spectra. This might be due to saturation of the available site on nanocrystal by biotin elements.

Thus, the various embodiments mentioned herein can be used for recognition of bio-molecules because of their selective influence on elements in biologic medium and detecting avidin concentration by measuring the emission-decreasing rate. It is seen that with reducing particle size, emission shifted to the lower wavelengths. In addition, with conjugation between avidin and biotin by mercaptoethanol in biologic media, spectral emission decreased. It is also seen that among the various concentrations of Mn⁺² ions (Manganese ion), a maximum emission is seen at an optimum doping at ˜5.5%. The fluorescence spectra of the doped crystals consist of orange-red emissions.

The foregoing description of the specific embodiments will so fully reveal the general nature of the embodiments herein that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A nano-biosensor for bimolecular recognition consisting of:
a nano particle of ZnS:Mn wherein the nano particle of ZnS:Mn comprises Zinc Sulphate hepta hydrate (ZnSO₄.7H₂O), Manganese nitrate tetrahydrate (Mn (NO₃)₂.4H₂O), Sodium sulphate, an oil, surfactant, co-surfactant, sodium hydroxide, linking agent and biotin.

2. The nano-biosensor according to claim 1, wherein the oil is cyclohexane.

3. The nano-biosensor according to claim 1, wherein the surfactant is triton X-100.

4. The nano-biosensor according to claim 1, wherein co-surfactant is n-hexanol.

5. The nano-biosensor according to claim 1, wherein the ratio of surfactant and co-surfactant is 1:1.

6. The nano-biosensor according to claim 1, wherein linking agents are mercaptoethanol and thioglycolic acid.

7. The nano-biosensor according to claim 1, wherein the linking agent is mercaptoethanol.

8. The nano-biosensor according to claim 1, wherein Mn(NO₃)₂.4H₂O has different concentrations and wherein different concentrations are 0.01%, 0.7%, 2%, 3%, 5.5%, 9% and 13%.

9. The nano-biosensor according to claim 1, wherein the size of nano particles of ZnS:Mn is 5-10 nm.

10. A method of synthesizing a nano-biosensor for bimolecular recognition consisting the steps of:
preparing a micro-emulsion solution by mixing cyclohexane, Triton X-100 and n-hexanol;
transferring the micro-emulsion solution into a first beaker and a second beaker wherein the amount of the micro-emulsion solution in the first beaker and the amount of the micro-emulsion solution in the second beaker are equal;
preparing an aqueous stock solution of ZnSO₄.7H₂O with a morality of 0.1;
preparing a pluralities of aqueous stock solutions of Mn(NO₃)₂.4H₂O with different concentrations and wherein the different concentrations are 0.01%, 0.7%, 2%, 3%, 5.5%, 9% and 13%;
preparing an aqueous stock solution of Na₂S with a morality of 0.1;
adding the aqueous stock solution of ZnSO₄.7H₂O and the pluralities of aqueous stock solutions of Mn(NO₃)₂.4H₂O with different concentrations to the first beaker;
agitating a content of the first beaker after adding the aqueous stock solution of ZnSO₄.7H₂O and the pluralities of aqueous stock solutions of Mn(NO₃)₂.4H₂O with different concentrations;
adding the aqueous stock solution of Na₂S to the second beaker;
agitating a content of the second beaker after adding the aqueous stock solution of Na₂S;
mixing the content of the first beaker and the content of the second beaker to obtain a resultant mixture;
agitating the resultant mixture;
keeping the resultant mixture at room temperature for aging;
adding a linking agent to the resultant mixture after aging; and
adding a biological agent to the resultant mixture to obtain a nano particle of ZnS:Mn.

11. The method according to claim 10, wherein the content of the first beaker is agitated by a magnetic stirrer for 15-120 min.

12. The method according to claim 10, wherein the content of the second beaker is agitated by a magnetic stirrer for 15-120 min.

13. The method according to claim 10, wherein the resultant mixture is agitated for 15 min.

14. The method according to claim 10, wherein cyclohexane is used as oil phase.

15. The method according to claim 10, wherein Triton X-100 is used as surfactant.

16. The method according to claim 10, wherein n-hexanol is used as co-surfactant.

17. The method according to claim 10, wherein the linking agents are mercaptoethanol and thioglycolic acid.

18. The method according to claim 10, wherein the linking agent is mercaptoethanol.

19. The method according to claim 10, wherein the biological agent is biotin.

20. The method according to claim 10, wherein the resultant mixture is kept at room temperature for 2 days for aging.
